(19) United States
(12) Patent Application Publication
(10) Pub. No.: US 2005/0240956 A1
(43) Pub. Date: Oct. 27, 2005

(54) Method and apparatus for enhancing wellness

(76) Inventors: Kurt Smith, Boulder, CO (US); Corwin Bell, Boulder, CO (US)

Correspondence Address: HOLLAND & HART, LLP, 555 17TH STREET, SUITE 3200, DENVER, CO, 80201, US

(21) Appl. No.: 10/830,778

(22) Filed: Apr. 22, 2004

(51) Int. Cl. H04N 7/16; A61B 5/00; G06K 9/00; H04H 9/00

(52) U.S. Cl. 725/10; 725/11; 382/115; 382/116

(57) ABSTRACT

The present invention provides methods and apparatuses to enhance wellness. In particular, the present invention provides a processor, a biofeedback sensor, and a wellness enhancing program that may comprise a series of multimedia events. The processor monitors a student's biometric information via the biofeedback sensor and uses the information to influence the wellness enhancing program.

### FIELD OF THE INVENTION

The present invention relates to wellness and, more particularly, methods and apparatuses to allow remote wellness coaching.

### BACKGROUND OF THE INVENTION

General emotional and physical wellness have been a concern for consumers for many years. Wellness products have emerged in the last decade to become a 100 billion dollar industry. Some wellness products include dietary products, such as, health food, multivitamins, etc., physical activity, such as, yoga and the like, and emotional care, such as, for example, counseling, meditation, and the like, or combinations thereof.

Personal wellness can be enhanced if a person learns how to control or influence their biometrics, such as, for example, heart rate, breathing, and the like. Measuring biometrics and learning how to control a body's response using a biofeedback paradigm helps reduce emotional and physical discomfort caused by stress, for example.

Learning how to control and regulate individual biometrics as a wellness tool frequently requires a student or client to attend a session with a psychiatrist, biofeedback technician, or other biofeedback coach. During these sessions, the student is provided direct feedback from the coach regarding the employed techniques.

However, using a coach typically requires traveling to an office or other facility so the student can meet with the coach. Driving to and from the office or other facility is inconvenient and can diminish the overall experience. Alternatively, some coaches visit a student's home, but this is expensive. Moreover, the personal coach is unavailable when traveling or if the student moves.

Thus, it would be desirous to provide methods and apparatuses to allow wellness coaching in the home of the user without having a live person (coach) visit the home.

### SUMMARY OF THE INVENTION

To attain the advantage of and in accordance with the purpose of the present invention, methods of for enhancing wellness are provided. The method comprises displaying multimedia events to a student at a local processor and monitoring biometric information of the student. The method further comprises using the monitored biometric information to modify the multimedia events displayed to the student.

The present invention further provides apparatuses to enhance wellness. The apparatus comprises a local processor accessible by a student. The student can access a wellness enhancing program. The apparatus further comprises a biofeedback sensor connected to the local processor to monitor the biometric information of the student. The biometric information gathered by the biofeedback sensor is used to influence the wellness enhancing program and for optional synchronous or asynchronous examination by a coach through a network connection.

The foregoing and other features, utilities and advantages of the invention will be apparent from the following more particular description of a preferred embodiment of the invention as illustrated in the accompanying drawings.

### BRIEF DESCRIPTION OF THE DRAWING

The accompanying drawings, which are incorporated in and constitute a part of this specification, illustrate embodiments of the present invention, and together with the description, serve to explain the principles thereof. Like items in the drawings are referred to using the same numerical reference.

FIG. 1 is a functional block diagram of a wellness system consistent with an embodiment of the present invention;

FIG. 2 is a flowchart illustrative of a wellness method consistent with an embodiment of the present invention;

FIG. 3 is a graphical representation of potential items displayable on a graphical user interface consistent with the present invention; and

FIG. 4 is a flowchart illustrative of a wellness method consistent with another embodiment of the present invention.

### DETAILED DESCRIPTION

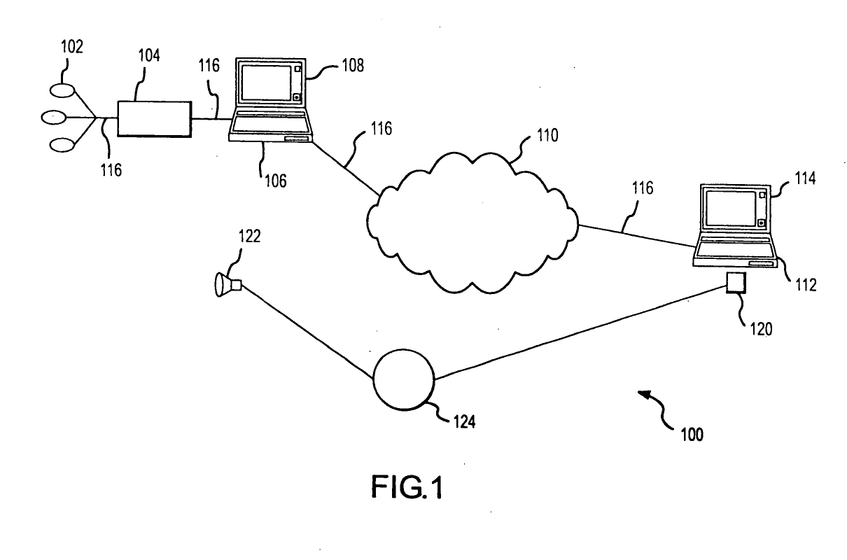

An embodiment of the present invention will be described with reference to FIGS. 1 to 4. As an initial matter, a coach teaches a client or student conventional biometric techniques. However, it is desirous to eliminate the physical presence of the coach altogether as a participant in the wellness session. The present invention provides apparatuses and methods to eliminate the coach by the use of a biofeedback device used in conjunction with specialized programming running on the client's local computer, or running on a remote processor and delivered to the client's local computer. The program allows the client himself to monitor and score the client's progress and provide the necessary multimedia feedback to encourage, direct and/or “coach” and score the client's progress. Alternatively, the program allows the coach to remotely monitor and score the client's progress and provide the necessary multimedia feedback to encourage, direct and/or “coach” and score the client's progress. It is well known that there exists a multitude of books, tapes, DVDs, and the like that provide self-coaching for wellness. However, the addition of biofeedback hardware and software interfaces in the client's home, and the ability of the client to monitor and score his or her progress using biofeedback is one difference between the existing books, tapes, DVDs, and the like and the present invention. Conventionally, this feedback was only possible through a biofeedback coach that provided the monitoring, coaching, and scoring. The present invention, however, enables wellness coaching, monitoring, feedback and scoring all within a local (or remote) program available within the client's home, desktop or laptop, computer or other processor. The present invention removes the live coach and provides a virtual coach, for lack of a better term, by biofeedback hardware that the client can use in the home or by remote coaching using a network connection. This provides the capability of effectively incorporating the biometrics from the biofeedback hardware into the “self-coached” wellness sessions. The results of the wellness sessions can be synchronously or asynchronously uploaded to a remote processor for record keeping, trend scoring, and analysis by a human wellness coach as necessary or desired.

Referring now to FIG. 1, FIG. 1 shows a functional block diagram of a wellness system 100 arranged in accordance with the present invention. Wellness system 100 comprises a biofeedback sensor 102, a biofeedback signal interface 104, a local processor 106, a local display 108, a network 110, a remote processor 112, and a remote display 114. Biofeedback sensor 102 is connected to biofeedback signal interface 104. Although only a single biofeedback sensor 102 is shown for convenience, local processor 106 could support multiple sensors and multiple users as desired.

Communication links 116 connect the various components of system 100. Communication links 116 could be any type of conventional connections, which include, for example, cable wire connections, fiber optic connections, wireless connections, bus connections, and the like as are generally known in the art and will not be further explained herein.

While only one local processor 106 is shown in FIG. 1, one of ordinary skill in the art would recognize that a number of local processors 106 could connect to remote processor 112 at any time. Further, local processor 106 and remote processor 112 could be single units as shown or a series of interconnected processors as a matter of design choice.

Local processor 106 and remote processor 112 can be any conventional processor, such as a desktop computer, laptop computer, server, PDA, cellular telephone, handheld computer, electronic game, conventional game platform, or the like, capable of connecting to network 110. Network 110 connecting local processor 106 and remote processor 112 could be any type of network such as a LAN, WAN, WLAN, intranet, Internet, World Wide Web, Ethernet, or the like. Using a web browser of the like to connecting multiple processors over a network to share data and/or resources is well known in the art and will not be specifically explained herein except to the extent necessary to explain the present invention.

Biofeedback sensor 102 could be, for example, a plurality of ring type sensors capable of fitting on the fingers of students. For example, two sensors may measure skin impedance. Measuring skin impedance provides an epidural skin response (also known as EDR or GSR) that is a useful indicator of, for example, the stress level of the student. Epidural skin response sensors are well known in the art and useful for devices, such as, lie detectors. Another sensor may be an infrared senor that may measure the student's heart rate. Infrared sensors of this sort are also well known in the art. Measuring the player's heartbeat can be useful in determining a variable heart rate. The variable heart rate could be useful in determining, for example, a coherence of the client's sympathetic and parasympathetic nervous system. While three sensors to determine two biometric values have been described, other combinations of sensors and other biometrics are possible. For example, biometrics could be measured for blood press, EKGs, EEGs, brain waves, blood oxygen levels, respiratory rates, or the like. Also, the wellness tool could be based on one or more biometric values measured using one or more biometric sensor. In other words, the combination of sensors and biometrics is a matter of design choice.

Biofeedback signal interface 104 converts the biometric values measured by biofeedback sensor and converts the biometric values into signals usable by local processor 106. Biofeedback signal interface 104 is shown as a separate component for convenience, but could be incorporated into biofeedback sensor 102 or local processor 106 as a matter of design choice.

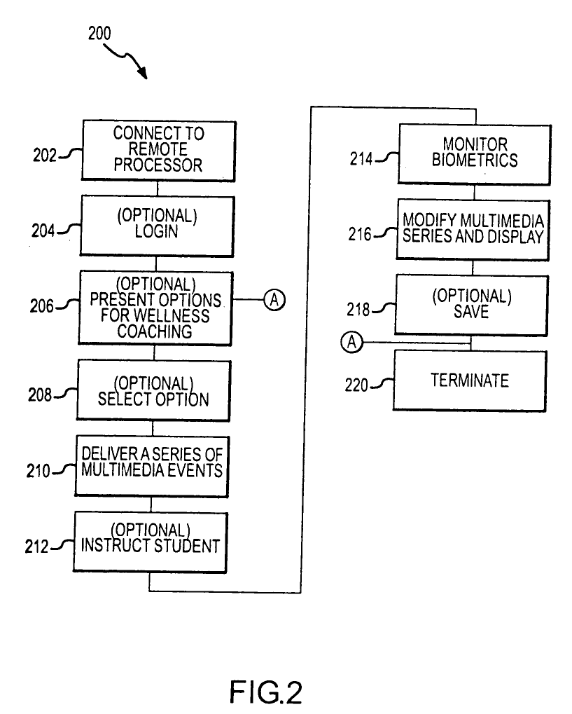

FIG. 2 shows a flowchart 200 illustrative of using system 100 as a wellness tool. First, using local processor 106, a student connects to remote processor 112 over the network 110, step 202. Local processor 106 could use a conventional web browser to connect with remote processor 112, or local processor 106 could execute specialized code to connect with remote processor 112. Once connected, the student optionally logs into a wellness program, step 204. Establishing an account facilitates maintaining records of the student's progress and billing, but logging in is not required for operation of system 100.

While a single wellness program is possible, it is envisioned that the student will be presented with a number of menu options for various wellness coaching categories, step 206. The student would select the option desired, such as, for example, a stress reduction session, step 208. A series of multimedia events would then be delivered to the student over network 110, step 210. The events could be batch loaded onto local processor 106 that would execute the program, streamed from processor 112 to processor 106, or using any conventional data transfer technology. Whether the actual exercise is run on processor 106, processor 112, or a combination thereof is a matter of design choice. Optionally, a tutorial or other instructional multimedia sequence could be used to instruct the student on the goals of the exercise and the biometric technique to be used, step 212. Further, while connection to remote processor 112 may be useful for record keeping, the actual software code for the wellness programs could be loaded on local processor 106 using conventional multimedia files, such as magnetic disks, optical disks, CDs, DVDs, or the like. Thus, instead of streaming or downloading multimedia files from remote processor 112 over network 110, multimedia files could be loaded locally using conventional techniques.

Once the exercise is selected and running, input from biofeedback sensor 102 is used by the processor (local processor 106 or remote processor 112) to monitor the student's biometrics, step 214. Based on the biofeedback signal, the multimedia series of events may be modified and displayed to the student, step 216. For example, if the multimedia event is related to stress reduction, and the goal is a calm sea, local display 108 may display a rough sea with lightening storm to indicate a relatively higher stress. As the biofeedback signal indicates the student entering a relatively lower stress level, the multimedia event may start displaying calmer seas and a drizzle. Other multimedia events could be used comprising images, audio, video or a combination thereof. The multimedia event(s) may include helpful options as known in the art, such as, for example, readme files, pull down menus, voice instructions, hyperlinks (internal and external links), videos, other graphics or audio, or the like, and combinations thereof. The exercise could continue until complete or the student interrupts the exercise, at which time the student may be given the option to save current progress, step 218, terminate the session, step 220, or return to the menu to select another option, step 206.

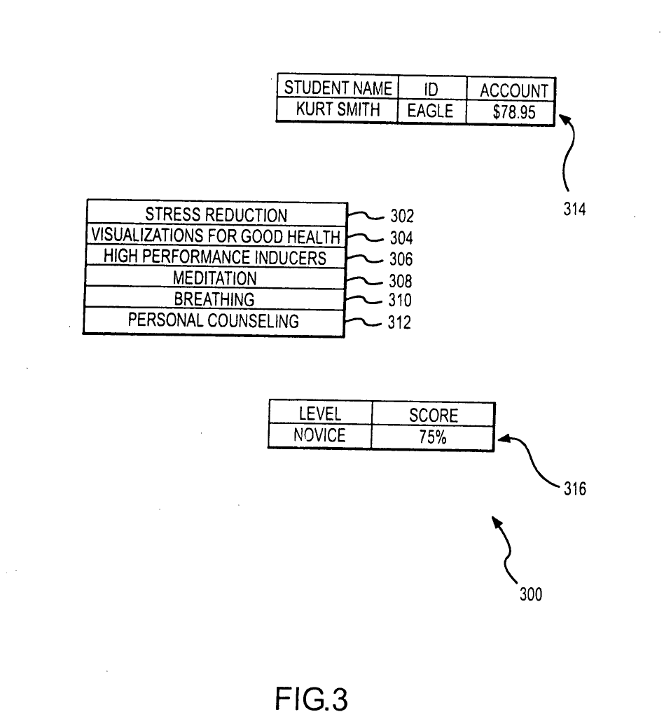

Referring to FIG. 3, a graphical user interface (GUI) 300 is provided to student at the menu selection screen. GUI 300 may provide a menu for stress reduction option 302, a visualizations for good health option 304, a high-performance inducers option 306, meditative options 308, breathing options 310, and personal counseling option 312 (which will be explained below). These, less, or other options are of course possible with the above being exemplary and non-limiting. GUI 300 could provide, as desired, other information as well, such as student accounting formation 314, student progress or score information 316, student records information, etc.

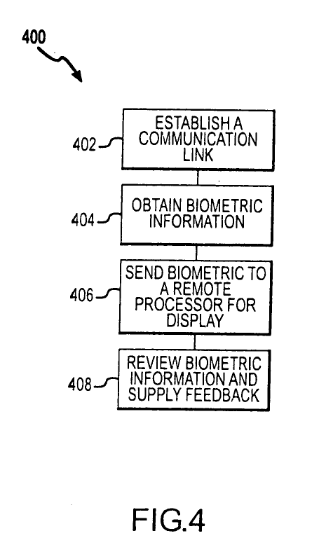

FIG. 4 is illustrates a flowchart 400 exemplary of a method by which a student at local processor 106 may receive personal counseling from a coach at remote processor 112. First, a communication link is established between local processor 106 and remote processor 112, step 402. Biometric information of the student is obtained by biofeedback sensor 102, step 404, and sent to remote processor 112 for display on remote display 114 such that the coach can view the biometric information, step 406. Biometrics could be similarly displayed on local display 108. The coach reviews the biometric information and provides feedback to local processor 106, step 408. The feedback could be transferred to local processor using any conventional technology, such as, a conventional chat room, email, instant messaging, or the like, conventional voice technology of the network, such as, VoIP or the like. Alternatively to establishing a connection via processor 112 and network 110 to processor 106, the coach could connect to the student using a conventional telephone connection. Referring back to FIG. 1, a remote telephone 120 could be connected to a local telephone 122 conventional telephone network 124, such as a conventional POTS system or a cellular system. In this case, part of the display for the coach would likely include a telephone number at which the student can be reached.

Generally, using the personal counseling session could be similar to the methodology provided by flowchart 200. With the personal coach, however, the coach could monitor biometrics and provide feedback via, an email, a text message, or a voice response (whether over a telephone or using voice synthesis technology via the computer).

While the invention has been particularly shown and described with reference to an embodiment thereof, it will be understood by those skilled in the art that various other changes in the form and details may be made without departing from the spirit and scope of the invention.

1. A computer implemented method for a student to be coached in biometric techniques, the method comprising the steps of:
establishing a communication link between a local processor and a remote processor;
transferring a series of multimedia events designed to teach biometric controlling techniques to the local processor over the established communication link;
displaying the series of multimedia events to the student;
monitoring biometric information of the student; and
modifying the series of multimedia events as necessary based on the biometric information of the student.

2. The method according to claim 1, wherein the step of establishing a communication link between the local processor and the remote processor includes the step of requiring the student to log in to the remote processor.

3. The method according to claim 1, wherein the established communication link is a two-way communication link.

4. The method according to claim 1, wherein step of transferring the series of multimedia events includes streaming multimedia event data from the remote processor to the local processor.

5. The method according to claim 1, wherein the step of monitoring biometric information includes streaming biometric information from the local processor to the remote processor.

6. The method according to claim 1, further comprising the step of transmitting coaching information to the student based on the monitored biometric information.

7. The method according to claim 1, further comprising
displaying a menu of a plurality of wellness options from which the student can select;
selecting one of the plurality of wellness options; and
wherein the step of transferring the series of multimedia events transfers the series of multimedia events based on the selected one of the plurality of wellness options.

8. The method according to claim 1, wherein the step of transferring the series of multimedia events includes downloading the series of multimedia events from the remote processor to the local processor and index information from the remote processor directs the display of the series of multimedia events to the student.

9. The method according to claim 1, further comprising the step of:
saving a progress of the student;

10. The method according to claim 1, wherein the step of displaying the series of multimedia events includes displaying a tutorial multimedia event.

11. The method according to claim 6, wherein the coaching information is transmitted by an email.

12. The method according to claim 6, wherein the coaching information is transmitted by a text message.

13. The method according to claim 6, wherein the coaching information is transmitted by VoIP.

14. The method according to claim 6, wherein the coaching information is transmitted by a telephone.

15. The method according to claim 6, wherein the coaching information is transmitted by a cellular telephone.

16. A computer implemented method of providing remote wellness care to a user, the method comprising the steps of:
connecting a local processor being used by a user to a remote processor containing wellness program options;
registering the user with the remote processor;
identifying to the user a plurality of wellness options;
selecting by the user at least one of the plurality of wellness options;
executing the at least one of the plurality of wellness options selected by the user, the executing step comprising:
providing a series of coaching instructions to the user; and
monitoring at least one biometric of the user.

17. The method according to claim 16 wherein the step of providing a series of coaching instructions comprises displaying a series of multimedia events to the user designed to coach the user in a biometric technique.

18. The method according to claim 16 wherein the step of providing a series of coaching instructions comprises providing verbal information from a personal coach.

19. The method according to claim 16 wherein the step of monitoring at least one biometric of the user comprises transmitting the at least one biometric to the remote processor.

20. The method according to claim 16 wherein the step of identifying to the user the plurality of wellness options comprises displaying a menu of the plurality of wellness options on a local display.

21. The method according to claim 16 wherein the step of registering the user includes the step of logging in at the remote processor.

22. The method according to claim 16, further comprising the steps of:
providing a communication link between a personal coach and the user, wherein the communication link is selected from the group consisting of a LAN, WAN, WLAN, Ethernet, Internet, Intranet, World Wide Web, POTS, VoIP, or cellular.

23. An apparatus for enhancing wellness, the apparatus comprising:
at least one local processor accessible by at least one user;
at least one remote processor;
at least one wellness enhancing program contained on the at least one remote processor;
at least one communication link between the at least one local processor and the at least one remote processor;
at least one biofeedback sensor connectable to the at least one user to sense biometric information from the at least one user; and
the at least one biofeedback sensor coupled to the at least one local processor such that the at least one local processor can receive biometric information from the at least one user, wherein
the at least one user can access the at least one wellness enhancing program from the at least one local processor and the biometric information of the at least one user can be used to influence the at least one wellness enhancing program.

24. The apparatus according to claim 23, wherein the at least one communication link is established to download the at least one wellness program.

25. The apparatus according to claim 24, wherein the biometric information is processed by the at least one local processor to influence the at least one wellness program.

26. The apparatus according to claim 23, wherein the at least one wellness program comprises streaming at least one multimedia event to the at least one local processor.

27. The apparatus according to claim 26, wherein the biometric information is streamed to the at least one remote processor over the at least one communication link to influence the at least one wellness program.

28. The apparatus according to claim 23, wherein the at least one communication link is a network selected from the group consisting of a local area network, a wide area network, a wireless network, a fiber network, a cable network, an Internet network, a World Wide Web network, an intranet, or an Ethernet.

29. The apparatus according to claim 23, further comprising
at least one coach monitoring biometric information at the at least one processor; and
at least a second communication link, the second communication link being between the at least one coach and the at least one user such that the at least one coach can provide feedback coaching information to the at least one user.

30. The apparatus according to claim 29, wherein the at least one communication link and the second communication link are the same.

31. The apparatus according to claim 29, wherein the at least one communication link and the second communication link are different.

32. The apparatus according to claim 31, wherein the second communication link is selected from the group consisting of a telephone connection, a wireless connection, an email connection, a text message connection, or an instant message connection.

33. An apparatus comprising:
at least one remote processor;
the at least one remote processor comprising a plurality of programs, wherein each of the plurality of programs provide at least one multimedia event designed to enhance the wellness of a student;
the at least one remote processor connectable to at least one local processor such that the at least one remote processor can transmit the at least one multimedia event to the student and receive biometric information from the student; and
means for modifying the at least one multimedia event being transmitted to the student based on the received biometric information.

34. An apparatus for enhancing wellness, the apparatus comprising:
at least one local processor accessible by at least one user;
the at least one local processor connectable to at least one remote processor;
at least one wellness enhancing program contained on the at least one local processor;
at least one communication link between the at least one local processor and the at least one remote processor;
at least one biofeedback sensor connectable to the at least one user to sense biometric information from the at least one user;
the at least one biofeedback sensor coupled to the at least one local processor such that the at least one local processor can receive biometric information from the at least one user;
the at least one user can access the at least one wellness enhancing program from the at least one local processor and the biometric information of the at least one user can be used to influence the at least one wellness enhancing program; and
a result from the at least one user accessing the at least one wellness enhancing program is transmitted from the at least one local processor to the at least one remote processor.

35. The apparatus according to claim 34, wherein the result is used by the at least one remote processor for accounting.

36. The apparatus according to claim 34, wherein the at least one wellness enhancing program can receive updated wellness enhancing programs from the at least one remote processor.

37. A computer implemented method for a student to be coached in biometric techniques, the method comprising the steps of:
accessing at least one multimedia event designed to teach biometric controlling techniques at a local processor;
displaying the at least one multimedia event to the student;
monitoring biometric information of the student;
modifying the at least one multimedia event based on the biometric information of the student; and
recording progress of the student at a remote processor.

38. The method of claim 37, wherein the recording progress of the student at a remote processor comprises the step of invoicing the student for access to the at least one multimedia event.

39. The method of claim 37, further comprising the step of:
loading the at least one multimedia event in the local processor.

40. The method of claim 39, wherein the step of loading the at least one multimedia event comprises reading the at least one multimedia event from a storage medium from the at least one local processor.

41. The method of claim 40, wherein the storage medium comprises storage selected from the group consisting of a hard drive, a magnetic disk, an optical disk, a DVD, and a CD.

42. The method of claim 39, further comprising the step of:
receiving updates to the at least one multimedia event from the at least one remote processor.

43. The method of claim 39, wherein the step of loading the at least one multimedia event comprises obtaining the at least one multimedia event from a storage medium from the at least one remote processor.

44. The method of claim 37, wherein the recording progress of the student at a remote processor comprises the step of recording a score for the student.

45. The method of claim 37, wherein the recording progress of the student at a remote processor comprises the step of recording a level for the student.

46. The method of claim 37, wherein step of displaying the at least one multimedia event to the student includes displaying a tutorial multimedia event.

47. The method of claim 6, wherein the step of transmitting coaching information is asynchronously transmitted.

48. The method of claim 6, wherein the step of transmitting coaching information is synchronously transmitted.

49. The method of claim 9, wherein the step of saving further comprises transmitting progress of the student to the remote processor asynchronously.

50. The method claim 9, wherein the step of saving further comprises transmitting progress of the student to the remote processor synchronously.

51. The method of claim 37, wherein the step of recording progress of the student comprises transmitting progress to the remote processor asynchronously.

52. The method of claim 37, wherein the step of recording progress of the student comprises transmitting progress to the remote processor asynchronously.

53. The method of claim 37, further comprising the step of
providing feedback to the student wherein the feedback is transmitted asynchronously.

54. The method of claim 53, wherein the step of providing feedback is based on recorded progress.

55. The method of claim 37, further comprising the step of providing feedback to the student wherein the feedback is transmitted synchronously.

56. The method of claim 55, wherein the step of providing feedback is based on recorded progress.

57. The method according to claim 1, wherein step of transferring the series of multimedia events includes streaming multimedia event index data from the remote processor to the local processor and using the multimedia event index data to display multimedia events to the student.

58. The method according to claim 6, wherein the coaching information is generated at the local processor.

59. The method according to claim 6, wherein the coaching information is generated at the remote processor.

60. A computer implemented method for a student to be coached in biometric techniques, the method comprising the steps of:
accessing a wellness enhancing program on a local processor, the wellness enhancing program containing a series of multimedia events designed to teach biometric controlling techniques;
displaying the series of multimedia events to the student;
monitoring biometric information of the student; and
analyzing the biometric information of the student and providing coaching information instructing the student on at least one technique to control at least one biometric.

61. The computer implemented method according to claim 60, wherein the step of analyzing the biometric information and providing coaching information instructing the student is accomplished by the local processor.

62. The computer implemented method according to claim 60, further comprising a communication link to a remote processor, wherein at least one of the steps of analyzing the biometric information and providing coaching information instructing the student is accomplished by the remote processor.

63. The computer implemented method according to claim 60, further comprising a communication link to a remote processor, wherein the remote processor provides information to the local processor regarding the series of multimedia events displayed to the student.

64. The computer implemented method according to claim 60, wherein the multimedia events are modified based on the monitored biometric information of the student.

65. The computer implemented method according to claim 60 wherein the student selects at least one wellness program from a plurality of wellness programs.

66. An apparatus for enhancing wellness, the apparatus comprising:
at least one local processor accessible by at least one user;
at least one wellness enhancing program contained on the at least one local processor, the at least one wellness enhancing program to display multimedia events to the at least one user;
at least one biofeedback sensor connectable to the at least one user to sense biometric information from the at least one user;
the at least one biofeedback sensor coupled to the at least one local processor such that the at least one local processor can receive biometric information from the at least one user; and
at least one biofeedback processor that receives the biometric information and provides coaching information to the at least one user designed to teach biometric controlling techniques, wherein
the at least one user can access the at least one wellness enhancing program from the at least one local processor and the biometric information of the at least one user is used to provide coaching information to the at least one user.

67. The apparatus according to claim 66, wherein the at least one biofeedback processor is integrated into the at least one local processor.

68. The apparatus according to claim 66, wherein the at least one biofeedback processor is remote from the at least one local processor.

69. The apparatus according to claim 68, wherein the at least one biofeedback processor is connected to the at leas tone local processor by a network connection.

70. The apparatus according to claim 66, wherein the at least one local processor is configured to receive updates over a network.
